(19) United States
(12) Patent Application Publication
(10) Pub. No.: US 2021/0321870 A1
(43) Pub. Date: Oct. 21, 2021

(54) RECOVERY MONITORING SYSTEM AND METHOD FOR HOSPITALIZED PATIENT, AND STORAGE MEDIUM

(71) Applicant: SHENZHEN MINDRAY BIO-MEDICAL ELECTRONICS CO., LTD., Shenzhen (CN)

(72) Inventors: Pengpeng NIE, Shenzhen (CN); Dayi MO, Shenzhen (CN); Bin LIU, Shenzhen (CN); Neng FU, Shenzhen (CN); Jun XU, Shenzhen (CN); Jian REN, Shenzhen (CN)

(73) Assignee: SHENZHEN MINDRAY BIO-MEDICAL ELECTRONICS CO., LTD., Shenzhen (CN)

(21) Appl. No.: 17/362,848

(22) Filed: Jun. 29, 2021

Related U.S. Application Data
(63) Continuation of application No. PCT/CN2018/125846, filed on Dec. 29, 2018.

(51) Int. Cl. A61B 5/00; H04Q 9/00

(52) U.S. Cl. CPC A61B 5/002; H04B 1/385; A61B 5/742; H04Q 9/00

(57) ABSTRACT

A recovery monitoring system for a hospitalized patient includes a ward-level monitoring apparatus at least comprising a first wireless communication module; and at least one mobile monitoring device attached to the body of a target object, where the at least one mobile monitoring device includes a second wireless communication module in coupling communication with the first wireless communication module. The at least one mobile monitoring device acquires a patient recovery state parameter of the target object, and uses a first wireless transmission scheme to transmit, by means of the second wireless communication module, the patient recovery state parameter to the first wireless communication module. The ward-level monitoring apparatus uses the first wireless transmission scheme to receive, by means of the first wireless communication module, the patient recovery state parameter from the at least one mobile monitoring device. The disclosed system realizes real time monitoring of a hospitalized patient.

### CROSS-REFERENCE TO RELATED APPLICATIONS

This application is a continuation of PCT Applications NO. PCT/CN2018/125846, file Dec. 29, 2018, which is incorporated herein by reference in its entirety.

### TECHNICAL FIELD

Embodiments of the disclosure relate to the technical field of medical instruments, and in particular to a patient recovery monitoring system and method applied in a hospital, and a storage medium.

### BACKGROUND

At present, for the intermediate link between severe patients and ordinary patients, major hospitals are committed to the construction of rehabilitation departments, that is, the construction of transitional sub-intensive care units. In a transitional sub-intensive care unit, during the rehabilitation of patients in sub-intensive care, the patients need more attention than those in a general ward, but less attention than those in an intensive care unit.

During the rehabilitation of the patients in sub-intensive care in a hospital, the patients do not stay in the bed all the time, but need to perform certain activities in the hospital to enhance the rehabilitation. In this process, real-time monitoring is required, that is, real-time monitoring and prompt of vital signs and other parameters of the patients in sub-intensive care, so as to avoid unexpected situations. However, for the traditional hospitalized monitoring, a bedside monitor is usually provided at the bedside of the patient to monitor and prompt in real time the parameters such as vital signs of the patient, so the patient is unable to move freely, and thus it is impossible to monitor the patient in sub-intensive care during activities.

### SUMMARY

In order to solve the above technical problems, embodiments of the present disclosure are expected to provide a patient recovery monitoring system and method applied in a hospital, and a storage medium. When a patient is moving inside and outside a ward, a patient recovery state parameter is obtained from a mobile monitoring device, and the patient recovery state parameter is transmitted to a ward-level monitoring apparatus to realize the monitoring of the patient.

The technical solutions of the embodiments of the disclosure may be implemented as follows.

An embodiment of the disclosure provides a patient recovery monitoring system applied in a hospital, the system comprising:

a ward-level monitoring apparatus comprising at least a first wireless communication module; and

at least one mobile monitoring device wearable on a body of a target object, where at least one of the at least one mobile monitoring device comprises a second wireless communication module capable of being coupled to and communicating with the first wireless communication module, and the at least one mobile monitoring device obtains a patient recovery state parameter corresponding to the target object, and transmits the patient recovery state parameter to the first wireless communication module in a first wireless transmission mode by means of the second wireless communication module; and

the ward-level monitoring apparatus receives the patient recovery state parameter from the at least one mobile monitoring device in the first wireless transmission mode by means of the first wireless communication module, so as to perform real-time monitoring on the target object in the hospital.

An embodiment of the disclosure provides a patient recovery monitoring system applied in a hospital, the system comprising:

a ward-level monitoring apparatus, a department-level monitoring apparatus and at least one mobile monitoring device, where

the at least one mobile monitoring device is capable of respectively transmitting a patient recovery state parameter with the ward-level monitoring apparatus and the department-level monitoring apparatus in a wireless transmission mode;

the at least one mobile monitoring device is wearable on a body of a target object, and is configured to obtain the patient recovery state parameter corresponding to the target object;

the at least one mobile monitoring device transmits the patient recovery state parameter detected in real time to the ward-level monitoring apparatus in a first wireless transmission mode; and

the at least one mobile monitoring device transmits the patient recovery state parameter detected in real time to the department-level monitoring apparatus in a second wireless transmission mode, where a transmission frequency band corresponding to the first wireless transmission mode is smaller than a transmission frequency band corresponding to the second wireless transmission mode.

An embodiment of the disclosure provides a patient recovery monitoring method applied in a hospital, which is applied to at least one mobile monitoring device, the method comprising:

obtaining a patient recovery parameter corresponding to a target object; and

transmitting the patient recovery state parameter to a ward-level monitoring apparatus in a first wireless transmission mode, such that the ward-level monitoring apparatus receives the patient recovery state parameter and performs real-time monitoring on the target object in the hospital.

An embodiment of the disclosure provides a patient recovery monitoring method applied in a hospital, which is applied to a patient monitoring apparatus, the method comprising:

receiving a patient recovery state parameter corresponding to a target object from at least one mobile monitoring device in a first wireless transmission mode, so as to perform real-time monitoring on the target object in the hospital,

where the at least one mobile monitoring device is configured to obtain the patient recovery state parameter.

An embodiment of the disclosure provides a mobile monitoring device, comprising: a first processor, a first memory, a first communication bus, a first communication accessory, and a detection accessory, where

the first communication bus is configured to implement a communication connection between the first processor, the first memory, the first communication accessory and the detection accessory; and

the first processor, the first communication accessory and the detection accessory are configured to execute a first monitoring procedure stored in the first memory to implement the above patient recovery monitoring method applied in a hospital, which is applied to a mobile monitoring device.

An embodiment of the disclosure provides a ward-level monitoring apparatus, comprising: a second processor, a second memory, a second communication bus and a second communication accessory, where

the second communication bus is configured to implement a communication connection between the second processor, the second memory and the second communication accessory; and

the second processor and the second communication accessory are configured to execute a second monitoring procedure stored in the second memory to implement the above patient recovery monitoring method applied in a hospital, which is applied to a ward-level monitoring apparatus.

An embodiment of the disclosure provides a computer-readable storage medium storing a first monitoring procedure, which is executable by a first processor, a first communication accessory and a detection accessory to implement the above patient recovery monitoring method applied in a hospital, which is applied to a mobile monitoring device.

An embodiment of the disclosure provides a computer-readable storage medium storing a second monitoring procedure, which is executable by a second processor and a second communication accessory to implement the above patient recovery monitoring method applied in a hospital, which is applied to a ward-level monitoring apparatus.

It can be seen therefrom that in the technical solution of the disclosure, a patient recovery monitoring system applied in a hospital comprises: a ward-level monitoring apparatus comprising at least a first wireless communication module; and at least one mobile monitoring device wearable on a body of a target object, where at least one of the at least one mobile monitoring device comprises a second wireless communication module capable of being coupled to and communicating with the first wireless communication module, and the at least one mobile monitoring device obtains a patient recovery state parameter corresponding to the target object, and transmits the patient recovery state parameter to the first wireless communication module in a first wireless transmission mode by means of the second wireless communication module; and the ward-level monitoring apparatus receives the patient recovery state parameter from the at least one mobile monitoring device in the first wireless transmission mode by means of the first wireless communication module, so as to perform real-time monitoring on the target object in the hospital. That is to say, when a patient is moving in the hospital, a patient recovery state parameter may be obtained from a mobile monitoring device, and the patient recovery state parameter is transmitted to a ward-level monitoring apparatus, such that the medical staff can check and learn the relevant condition of the patient at any time to realize the monitoring of the patient.

### BRIEF DESCRIPTION OF THE DRAWINGS

FIG. 1 is structural schematic diagram I of a patient recovery monitoring system applied in a hospital according to an embodiment of the disclosure;

FIG. 2 is a structural schematic diagram of an exemplary mobile monitoring device and ward-level monitoring apparatus according to an embodiment of the disclosure;

FIG. 3 is a system schematic diagram of an exemplary patient recovery monitoring system applied in a hospital according to an embodiment of the disclosure;

FIG. 4 is a schematic diagram in which an exemplary mobile monitoring device is worn according to an embodiment of the disclosure;

FIG. 5 is a schematic diagram of a topological structure of an exemplary first wireless communication module according to an embodiment of the disclosure;

FIG. 6 is a schematic diagram of a topological structure of an exemplary second wireless communication module according to an embodiment of the disclosure;

FIG. 7 is a structural schematic diagram II of a patient recovery monitoring system applied in a hospital according to an embodiment of the disclosure;

FIG. 8 is a structural schematic diagram III of a patient recovery monitoring system applied in a hospital according to an embodiment of the disclosure;

FIG. 9 is a structural schematic diagram IV of a patient recovery monitoring system applied in a hospital according to an embodiment of the disclosure;

FIG. 10 is a structural schematic diagram of an exemplary mobile monitoring device according to an embodiment of the disclosure;

FIG. 11 is a structural schematic diagram V of a patient recovery monitoring system applied in a hospital according to an embodiment of the disclosure;

FIG. 12 is a communication schematic diagram of an exemplary master mobile monitoring device and slave mobile monitoring device according to an embodiment of the disclosure;

FIG. 13 is a communication schematic diagram of an exemplary master mobile monitoring device, slave mobile monitoring device and ward-level monitoring apparatus according to an embodiment of the disclosure;

FIG. 14 is a structural schematic diagram of an exemplary bedside monitor according to an embodiment of the disclosure;

FIG. 15 is a communication schematic diagram of an exemplary extended monitoring device according to an embodiment of the disclosure;

FIG. 16 is a flow schematic diagram of a patient recovery monitoring method according to an embodiment of the disclosure;

FIG. 17 is a structural schematic diagram of a mobile monitoring device according to an embodiment of the disclosure; and

FIG. 18 is a structural schematic diagram of a ward-level monitoring apparatus according to an embodiment of the disclosure.

### DETAILED DESCRIPTIONS

The description has been made with reference to various exemplary embodiments herein. However, those skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

The terms “first”, “second” and the like in the specification and the claims of the disclosure and the above drawings are used to distinguish different objects, but not to describe a specific order. In addition, the terms “comprise”, “have”, and any variations thereof are intended to cover non-exclusive inclusion. For example, a process, a method, a system, a product, or a device that includes a series of steps or units is not limited to the listed steps or units, but optionally further includes unlisted steps or units, or optionally further includes other steps or units inherent in these processes, methods, or devices.

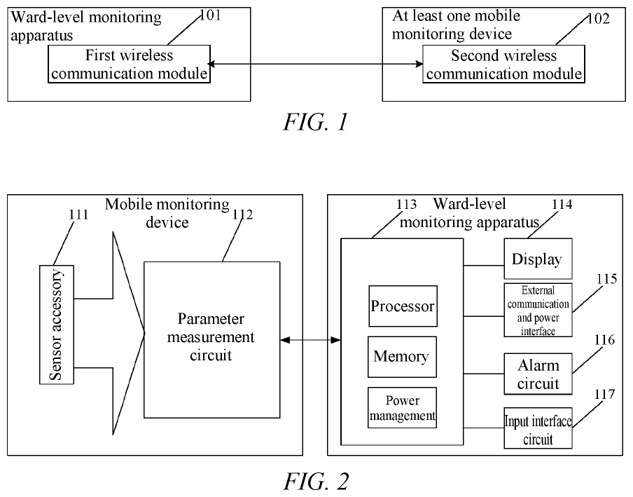

An embodiment of the disclosure provides a patient recovery monitoring system applied in a hospital. FIG. 1 is structural schematic diagram I of a patient recovery monitoring system applied in a hospital according to an embodiment of the disclosure. As shown in FIG. 1, the system comprises:

a ward-level monitoring apparatus comprising at least a first wireless communication module 101; and

at least one mobile monitoring device wearable on a body of a target object. At least one of the at least one mobile monitoring device comprises a second wireless communication module 102 capable of being coupled to and communicating with the first wireless communication module 101. The at least one mobile monitoring device obtains a patient recovery state parameter corresponding to the target object, and transmits the patient recovery state parameter to the first wireless communication module 101 in a first wireless transmission mode by means of the second wireless communication module 102.

The ward-level monitoring apparatus receives the patient recovery state parameter from the at least one mobile monitoring device in the first wireless transmission mode by means of the first wireless communication module 101, so as to perform real-time monitoring on the target object in the hospital.

It should be noted that in an embodiment of the disclosure, the patient recovery parameter includes at least one of the following three types of parameters:

parameters related to movement amount, such as movement steps, step frequency, movement distance, calories, etc.;

a physiological parameter, such as blood oxygen, blood pressure, pulse rate, body temperature, ECG, respiration, and other parameters, as well as related statistics and a rate of change of these parameters; and

time parameters of the human body state, for example, a movement-related or sleep-related time parameter that characterizes a human body state, such as a sleep time or a movement time. The specific patient recovery state parameter is not limited in this embodiment of the disclosure.

Accordingly, as shown in FIG. 2, the mobile monitoring device further comprises at least a parameter measurement circuit 112. The parameter measurement circuit 112 comprises at least a parameter measurement circuit corresponding to a physiological parameter. The parameter measurement circuit includes at least one of: an ECG signal parameter measurement circuit, a respiration parameter measurement circuit, a body temperature parameter measurement circuit, a blood oxygen parameter measurement circuit, a non-invasive blood pressure parameter measurement circuit, an invasive blood pressure parameter measurement circuit, etc. Each parameter measurement circuit is connected to an externally inserted sensor accessory 111 via a corresponding sensor interface. The sensor accessory 111 comprises detection accessories corresponding to the detection of physiological parameters such as ECG, respiration, blood oxygen, blood pressure and body temperature. The parameter measurement circuit is mainly used for connecting the sensor accessory 111 to obtain collected physiological parameter signals, and may comprise measurement circuits for at least two physiological parameters. The parameter measurement circuit 112 may be, but is not limited to, a physiological parameter measurement circuit (module), a human physiological parameter measurement circuit (module), a sensor for collecting a human physiological parameter, etc.

The ward-level monitoring apparatus further comprises a master control circuit, which necessarily comprises at least one processor and at least one memory. Of course, the master control circuit may further comprise at least one of: a power management module, a power IP module, an interface conversion circuit, etc. The power management module is configured to control the power on and off of an entire machine, a power-on sequence of each power domain inside a board card, and battery charging and discharging. The power IP module is a separate power module firmed by associating a principle diagram of a power supply circuit unit that is frequently and repeatedly invoked, with a PCB layout. That is, an input voltage is converted into an output voltage by means of a predetermined circuit. The input voltage and the output voltage are different. For example, the voltage of 15 V is converted into 1.8 V, 3.3 V, 3.8 V, etc. It can be understood that the power IP module may be single-channel or multi-channel. When the power IP module is single-channel, the power IP module can convert one input voltage into one output voltage. When the power IP module is multi-channel, the power IP module can convert one input voltage into a plurality of output voltages, and voltage values of the plurality of output voltages may be the same or different, such that different voltage requirements of a plurality of electronic components can be met at the same time. In addition, the module has few external interfaces, and works in the system as a black box decoupled from an external hardware system, which improves the reliability of the entire power system. The interface conversion circuit is configured to convert a signal output by a master control minimum system module (i.e., at least one processor and at least one memory in the master control circuit) into an input standard signal required to be received by an actual external device. For example, supporting an external VGA display function is to convert an RGB digital signal output by a master control CPU into a VGA analog signal, and supporting an external network function is to convert an RMII signal into a standard network differential signal.

Furthermore, the ward-level monitoring apparatus may further comprise one or more of: a local display 114, an alarm circuit 116, an input interface circuit 117, and a power interface 115. The master control circuit is configured to coordinate and control board cards, circuits and devices in the multi-parameter monitor or module assembly. In this embodiment, in conjunction with FIG. 1, with reference to FIG. 2, the master control circuit is configured to control data interaction between the first wireless communication module 101 and the second wireless communication module 102, and control the transmission of a control signal and transfer the physiological data to the display 114 for displaying, and may also receive user control instructions input from a touch screen or a physical input interface circuit such as a keyboard and keys, and of course, may also output a control signal on how to collect the physiological parameter. The alarm circuit 116 may be an audible and visual alarm circuit. The first wireless transmission mode may be one of or a combination of: wireless interfaces of infrared, Bluetooth, WI-FI, WMTS communication, etc.

It should be noted that in an embodiment of the disclosure, the transmission frequency band corresponding to the first wireless transmission mode is not more than 1 GHz, or the first wireless transmission mode uses a medical professional frequency band. That is to say, the first wireless transmission mode may actually be a Wireless Medical Telemetry Services (WMTS) transmission mode.

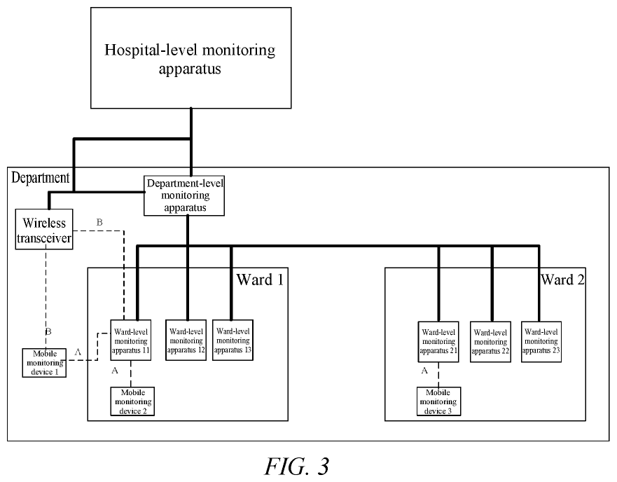

FIG. 3 is a system schematic diagram of an exemplary patient recovery monitoring system applied in a hospital according to an embodiment of the disclosure. As shown in FIG. 3, the patient recovery monitoring system may be a three-level monitoring system comprising a ward-level monitoring apparatus, a department-level monitoring apparatus and a hospital-level monitoring apparatus. With this system, the data of the monitor can be saved as a whole, patient information and nursing information can be managed centrally and stored in association, which facilitates the storage of historical data and associated alarming. One bed-level monitoring apparatus, for example, a bedside monitor, may be provided for each hospital bed. After the wireless communication between the mobile monitoring device and the bed-level monitoring apparatus, the patient recovery state parameter obtained from the mobile monitoring device can be transmitted to the bed-level monitoring apparatus for displaying or to a department-level monitoring apparatus or even a hospital-level monitoring apparatus of the hospital bed for viewing by a doctor or a nurse, or transmitted to a data server by means of a bed-level monitoring apparatus for storage. In addition, with the mobile monitoring device, the patient recovery state parameter generated by a mobile monitor may also be transmitted, by means of a wireless network node arranged in the hospital, to a department-level monitoring apparatus or a hospital-level monitoring apparatus of the hospital bed for storage and displaying, or the patient recovery state parameter obtained from the mobile monitoring device may be transmitted, by means of the wireless network node arranged in the hospital, to the data server for storage. It can be seen that the data corresponding to the patient recovery state parameter displayed on the ward-level monitoring apparatus can be derived from the sensor accessory directly connected to the monitor, or derived from the mobile monitoring device, or derived from the data server.

The at least one mobile monitoring device described above transmits, in a first wireless transmission mode, the patient recovery state parameter to the ward-level monitoring apparatus, which is a first level of monitoring system. That is, in FIG. 3, the mobile monitoring device 2 can communicate with the ward-level monitoring apparatus 11, with the communication mode A therebetween being a first wireless transmission mode, that is, the WMTS transmission mode.

In an embodiment of the disclosure, at least one mobile monitoring device is worn at any position on the body of the target object, and preferably on one or more of: a wrist, a leg, an arm, a chest, a finger and a waist of the target object.

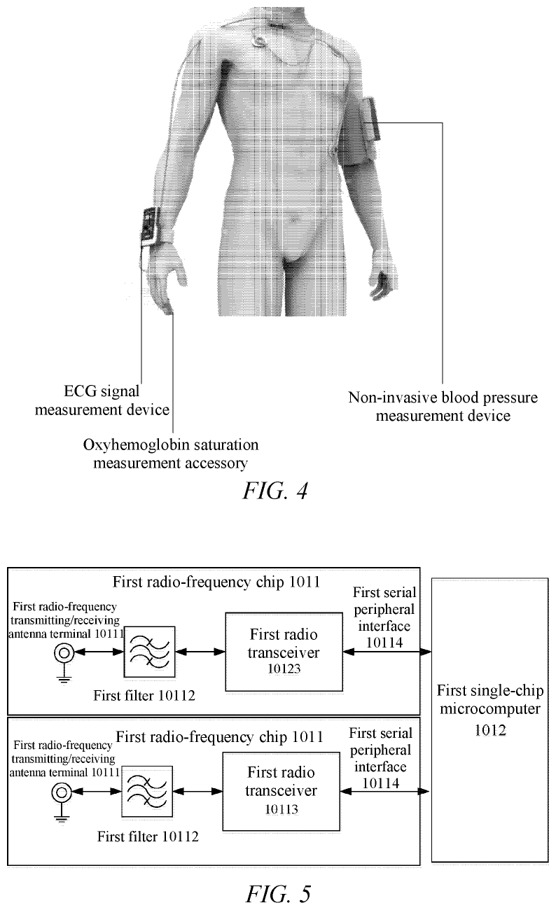

FIG. 4 is a schematic diagram in which an exemplary mobile monitoring device is worn according to an embodiment of the disclosure. As shown in FIG. 4, the body of the target object is worn with two mobile monitoring devices: an ECG signal measurement device and a non-invasive blood pressure measurement device. The ECG signal measurement device (referred to as ECG-PoD below) may be wearable on the wrist, and the non-invasive blood pressure measurement device (referred to as NIBP-PoD below) may be wearable on the arm.

It should be noted that at least one of the at least one mobile monitoring device is connected to an external parameter measurement accessory in a wired or wireless mode, and the external parameter measurement accessory is attached to a part of the body of the target object that corresponds to a parameter to be measured. The specific external parameter measurement accessory is not limited in the embodiment of the disclosure.

As an example, in an embodiment of the disclosure, as shown in FIG. 4, the at least one mobile monitoring device includes an ECG-PoD, and the ECG-PoD is connected to the external parameter measurement accessory in a wired connection mode. In this embodiment, the external parameter measurement accessory comprises an oxyhemoglobin saturation measurement accessory configured to measure the oxyhemoglobin saturation of the target object.

It should be noted that in an embodiment of the disclosure, before the at least one mobile monitoring device transmits the patient recovery state parameter to the ward-level monitoring apparatus in the first wireless transmission mode, the two also need the pairing with therebetween.

In an embodiment of the disclosure, the ward-level monitoring apparatus broadcasts identification information of the ward-level monitoring apparatus at a fixed frequency by means of the first wireless communication module 101; and at least one mobile monitoring device scans the fixed frequency by means of the second wireless communication module 102 to receive the identification information of the ward-level monitoring apparatus, and synchronously sets the fixed frequency according to the identification information of the ward-level monitoring apparatus, to realize the pairing with the ward-level monitoring apparatus.

It should be noted that in an embodiment of the disclosure, the pairing of the ward-level monitoring apparatus and the at least one mobile monitoring device can be implemented not only by means of broadcasting the identification information of the ward-level monitoring apparatus and setting the fixed frequency as described above, but also by means of Near Field Communication (NFC) to implement pairing. Specifically, it is only necessary to configure corresponding communication modules for the ward-level monitoring apparatus and the mobile monitoring device, and when the two touch in a short distance, the frequencys can be automatically paired, WMTS can be connected, and the patient recovery state parameter can be transmitted by means of WMTS.

It will be understood that in an embodiment of the disclosure, in the at least one mobile monitoring device, each mobile monitoring device can be paired with the ward-level monitoring apparatus or, of course, only some mobile monitoring devices are paired with the ward-level monitoring apparatus. For example, one mobile monitoring device is selected from the at least one mobile monitoring device and is paired with the ward-level monitoring apparatus, and the paired mobile monitoring device can collect the patient recovery state parameter obtained from the at least one mobile monitoring device. The specific obtaining mode will be described in detail below.

In an embodiment of the disclosure, the first wireless communication module 101 comprises at least one first radio-frequency module 1011 and a first single-chip microcomputer 1012; and the second wireless communication module 102 comprises a second radio-frequency module 1021 and a second single-chip microcomputer 1022.

The second wireless communication module 102 transmits the patient recovery state parameter to the second radio-frequency module 1021 by means of the second single-chip microcomputer 1022, and transmits the patient recovery state parameter to the first wireless communication module 101 by means of the second radio-frequency module 1021.

The first wireless communication module 101 receives the patient recovery state parameter by means of the at least one first radio-frequency module 1011 to obtain at least one set of patient recovery state parameters, and selects one set of data from the at least one set of patient recovery state parameters by means of the first single-chip microcomputer 1012 so as to implement the real-time monitoring.

It will be understood that in an embodiment of the disclosure, the first wireless communication module 101 receives, by means of the at least one first radio-frequency module 1011, at least one set of patient recovery state parameters, from which the first single-chip microcomputer 1012 can select one set of data with higher accuracy, so as to ensure the accuracy of the patient recovery state parameters obtained from the ward-level monitoring apparatus.

Specifically, in an embodiment of the disclosure, the at least one first radio-frequency module 1011 comprises at least one first radio-frequency transmitting/receiving antenna terminal 10111, at least one first filter 10112, at least one first radio transceiver 10113, and at least one first serial peripheral interface 10114; and the second radio-frequency module 1021 comprises a second radio-frequency transmitting/receiving antenna terminal 10211, a second filter 10212, a second radio transceiver 10213, and a second serial peripheral interface 10214.

The second radio-frequency module 1021 receives the patient recovery state parameter by means of the second serial peripheral interface 10214, and transmits the patient recovery state parameter to the at least one first radio-frequency module 1011 by means of the second radio transceiver 10213, the second filter 10212 and the second radio-frequency transmitting/receiving antenna terminal 10211 in sequence.

The at least one first radio-frequency module 1011 receives the patient recovery state parameter by means of the at least one first radio-frequency transmitting/receiving antenna terminal 10111, the at least one first filter 10112 and the at least one first radio transceiver 10113 in sequence, and transmits the patient recovery state parameter to the first single-chip microcomputer 1012 by means of the at least one first serial peripheral interface 10114.

FIG. 5 is a schematic diagram of a topological structure of an exemplary first wireless communication module according to an embodiment of the disclosure. As shown in FIG. 5, the first wireless communication module 101 comprises two first radio-frequency modules 1011 and one first single-chip microcomputer 1012. Each of the first radio-frequency modules 1011 comprises one first radio-frequency transmitting/receiving antenna terminal 10111, one first filter 10112, one first radio transceiver 10113 and one first serial peripheral interface 10114.

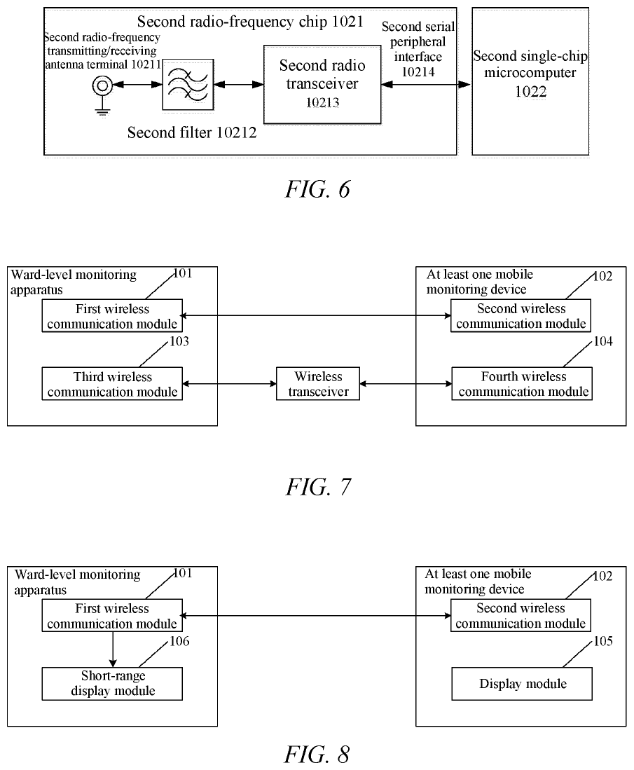

FIG. 6 is a schematic diagram of a topological structure of an exemplary second wireless communication module according to an embodiment of the disclosure. As shown in FIG. 6, the second wireless communication module 102 may only comprise one second radio-frequency module 1021 and one second single-chip microcomputer 1022, and the second radio-frequency module 1021 specifically comprises a second radio-frequency transmitting/receiving antenna terminal 10211, a second filter 10212, a second radio transceiver 10213 and a second serial peripheral interface 10214.

It will be understood that in an embodiment of the disclosure, as shown in FIGS. 5 and 6, in order to improve the reliability of the uplink, the ward-level monitoring apparatus uses two first radio-frequency modules to respectively receive the patient recovery state parameter so as to obtain two sets of patient recovery state parameters and selects, from the two sets of patient recovery state parameters, the set of data which is accurate by means of the single-chip microcomputer, while the mobile monitoring device only needs to use one second radio-frequency module to issue the patient recovery state parameter. Of course, if the ward-level monitoring apparatus is required to issue the patient recovery state parameter to the mobile monitoring device or other apparatuses, only any one of the two first radio-frequency modules is required to operate, instead of the two first radio-frequency modules operating simultaneously. That is to say, the ward-level monitoring apparatus and the mobile monitoring device maintain a half-duplex operating mode, which can implement bidirectional communication.

It should be noted that in an embodiment of the disclosure, not only two first radio-frequency modules 1011 are configured for the first wireless communication module 101, but one first radio-frequency module 1011 can also be reserved, i.e., configuring three first radio-frequency modules 1011 for scanning the spatial signal intensity. It is possible to further extend and add radio-frequency modules for transmission and evaluation of channel usage.

FIG. 7 is structural schematic diagram II of a patient recovery monitoring system applied in a hospital according to an embodiment of the disclosure. As shown in FIG. 7, the patient recovery monitoring system further comprises: a wireless transceiver. The ward-level monitoring apparatus further comprises a third wireless communication module 103 that can access the wireless transceiver. At least one of the at least one mobile monitoring device comprises a fourth wireless communication module 104 that can access the wireless transceiver. The ward-level monitoring apparatus accesses the wireless transceiver by means of the third wireless communication module 103, and the at least one mobile monitoring device accesses the wireless transceiver by means of the fourth wireless communication module 104, so as to transmit the patient recovery state parameter from the at least one mobile monitoring device to the ward-level monitoring apparatus in the second wireless transmission mode by means of the wireless transceiver. A transmission frequency band corresponding to the first wireless transmission mode is less than a transmission frequency band corresponding to the second wireless transmission mode. It will be understood that in other embodiments of the disclosure, the transmission frequency band corresponding to the first wireless transmission mode may also be equal to or greater than the frequency band corresponding to the second wireless transmission mode.

It should be noted that in an embodiment of the disclosure, the second wireless transmission mode may be a Wireless-Fidelity (WI-FI) mode, and may also be a WMTS transmission mode, which is the same as the first wireless transmission mode. The specific second wireless transmission mode is not limited in the embodiment of the disclosure.

It will be understood that in an embodiment of the disclosure, since the second wireless transmission mode may be a WI-FI transmission mode or may be a WMTS transmission mode, the wireless transceiver includes one of the following: a WI-FI wireless transceiver; and a wireless transceiver operating in a medical professional frequency band of a second operating frequency band, wherein the second operating frequency band being greater than a first operating frequency band which is a medical professional frequency band not more than 1 GHz.

It should be noted that in an embodiment of the disclosure, the wireless transceiver not only can serve as a WI-Fi transceiver to implement WI-FI access of the ward-level monitoring apparatus and the mobile monitoring device, but also can form another WMTS frequency band, i.e., the second operating frequency band that is greater than the first operating frequency band, which can meet the longer-distance WMTS communication. Moreover, the first wireless transmission mode between the above ward-level monitoring apparatus and the at least one mobile monitoring device uses the first operating frequency band, which is adapted to the WMTS communication in the ward, while the second operating frequency band used by the wireless transceiver is adapted to the WMTS communication outside the ward. It will be understood that other modes can be used to enable the transmission distance of the second wireless transmission mode to be greater than the first wireless transmission mode.

As an example, in an embodiment of the disclosure, as shown in FIG. 3, the mobile monitoring device 1 may comprise a fourth wireless communication module 104, and accesses the wireless transceiver by means of the fourth wireless communication module 104. Similarly, the ward-level monitoring apparatus 11 comprises a third wireless communication module 103, and may also access the wireless transceiver. B can be indicative of WI-FI transmission mode, i.e., the mobile monitoring device 1 and the ward-level monitoring apparatus 11 can transmit the patient recovery state parameter in the WI-FI transmission mode.

It will be understood that in an embodiment of the disclosure, if the mobile monitoring device is far away from the ward-level monitoring apparatus, it may be difficult to perform normal data transmission using the first wireless transmission mode. Therefore, at least one of the at least one mobile monitoring device accesses the wireless transceiver by means of the fourth wireless communication module 104, and similarly, the ward-level monitoring apparatus accesses the wireless transceiver by means of the third wireless communication module 103 configured by itself, which can implement longer-distance transmission of the patient recovery state parameter to solve the problem of long-distance transmission of the patient recovery state parameter.

FIG. 8 is structural schematic diagram III of a patient recovery monitoring system applied in a hospital according to an embodiment of the disclosure. As shown in FIG. 8, the at least one mobile monitoring device comprises at least one display module 105, and the ward-level monitoring apparatus comprises at least one nearby display module 106.

The at least one mobile monitoring device obtains the patient recovery state parameter corresponding to the target object to form local display data, and outputs and displays same on the display module 105, with the local display data including a numerical value and/or a waveform.

The ward-level monitoring apparatus obtains the patient recovery state parameter by means of the first wireless communication module 101 to form a nearby display data, and outputs and displays same on the nearby display module 106, with the nearby display data including a numerical value and/or a waveform.

It should be noted that in an embodiment of the disclosure, any one of the at least one mobile monitoring device may be configured with the display module 105. The patient recovery state parameter, when being obtained, can be output and displayed on any one of the display modules 105, and may be directly displayed as corresponding specific numerical values, and may also be displayed as waveforms formed by the numerical values, which are all local display data. The specific display mode is not limited in the embodiment of the disclosure. It will be understood that the mobile monitoring apparatus may further comprise an information processing module configured to process the physiological parameter detected by the parameter measurement circuit. At the same time, when the mobile monitoring apparatus comprises the information processing module and the display module 105, the parameter measurement circuit may also be integrated on the sensor accessory that is connected to the parameter measurement circuit.

It should be noted that in an embodiment of the disclosure, after the ward-level monitoring apparatus obtains the patient recovery state parameter, data processing can be performed on the patient recovery state parameter to obtain a data processing result, and a nearby display data is formed according to the data processing result and the patient recovery state parameter. Therefore, the numerical value and/or the waveform shown by the nearby display module 106 may be different from the local display data.

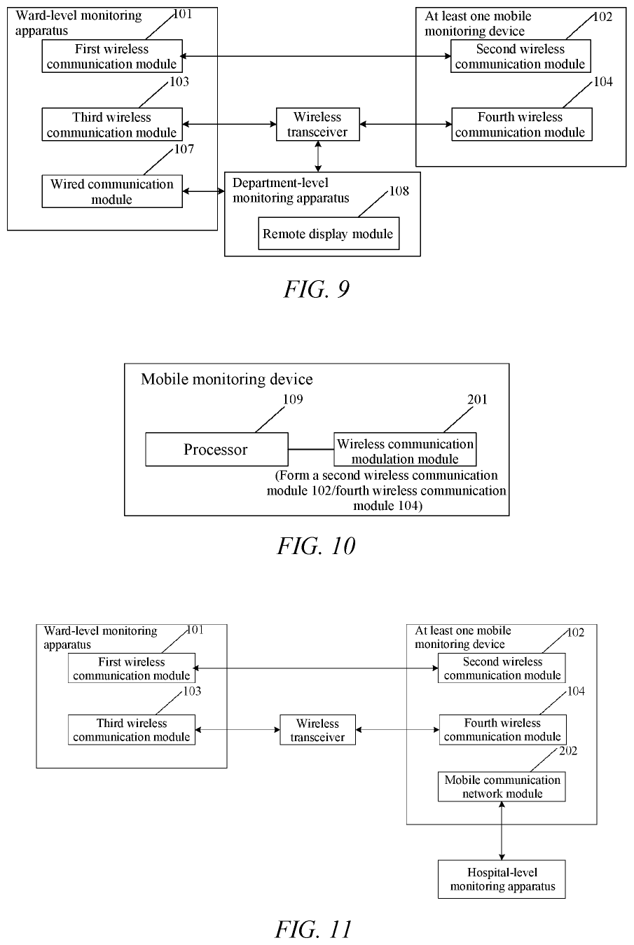

FIG. 9 is a structural schematic diagram IV of a patient recovery monitoring system applied in a hospital according to an embodiment of the disclosure. As shown in FIG. 9, the patient recovery monitoring system may further comprise: a department-level monitoring apparatus; the ward-level monitoring apparatus may transmit the patient recovery state parameter to the department-level monitoring apparatus by means of a built-in wired communication module 107; and/or the department-level monitoring apparatus is communicatively connected to the wireless transceiver, and at least one mobile monitoring device accesses the wireless transceiver by means of the fourth wireless communication module 104, so as to transmit the patient recovery state parameter from the at least one mobile monitoring device to the department-level monitoring apparatus in the second wireless transmission mode by means of the wireless transceiver.

Specifically, in an embodiment of the disclosure, as shown in FIG. 3, the department-level monitoring apparatus manages a ward 1 and a ward 2. The ward 1 comprises: a ward-level monitoring apparatus 11, a ward-level monitoring apparatus 12 and a ward-level monitoring apparatus 13. The ward 2 comprises: a ward-level monitoring apparatus 21, a ward-level monitoring apparatus 22 and a ward-level monitoring apparatus 23. The ward-level monitoring apparatus in either of the ward 1 and the ward 2 is connected to the department-level monitoring apparatus in a wired or WI-FI mode, and therefore the ward-level monitoring apparatus in either of the ward 1 and the ward 2 can forward the received patient recovery state parameter to the department-level monitoring apparatus by means of the built-in wired communication module 107.

Specifically, in an embodiment of the disclosure, as shown in FIG. 9, the department-level monitoring apparatus is in wired communication connection with the wireless transceiver. Therefore, after the mobile monitoring device accesses the wireless transceiver by means of the fourth wireless communication module 104, the patient recovery state parameter can not only be transmitted to the ward-level monitoring apparatus in the second wireless transmission mode, but also transmitted to the department-level monitoring apparatus.

In an embodiment of the disclosure, at least one mobile monitoring device comprises at least one display module 105, the system further comprises a department-level monitoring apparatus, and the department-level monitoring apparatus comprises at least one remote display module 108.

The at least one mobile monitoring device obtains the patient recovery state parameter corresponding to the target object to form local display data, and outputs and displays same on the display module 105, with the local display data including a numerical value and/or a waveform.

The department-level monitoring apparatus uses the second wireless transmission mode to obtain the patient recovery state parameter to form remote display data, and outputs and displays same on the remote display module 108, with the remote display data including a numerical value and/or a waveform.

It will be understood that in an embodiment of the disclosure, similar to the above ward-level monitoring apparatus, the department-level monitoring apparatus may also be configured with a remote display module 108, and the department-level monitoring apparatus may also form remote display data according to the patient recovery state parameter and display same, which will not be described in detail herein.

FIG. 10 is a structural schematic diagram of an exemplary mobile monitoring device according to an embodiment of the disclosure. As shown in FIG. 10, in an embodiment of the disclosure, at least one of the at least one mobile monitoring device comprises a processor 109 and a wireless communication modulation module 201. The processor 109 causes the wireless communication modulation module 201 to operate in a first operating frequency band to form a second wireless communication module 102, such that the mobile monitoring device transmits the patient recovery state parameter to the first wireless communication module 101 in the first operating frequency band by means of the second wireless communication module 102.

When a preset condition is satisfied, the processor 109 causes the wireless communication modulation module 201 to switch to a second operating frequency band to form a fourth wireless communication module 104, such that the at least one mobile monitoring device performs wireless transmission of the patient recovery state parameter in the second operating frequency band by means of the fourth wireless communication module 104, with the second operating frequency band being greater than the first operating frequency band. The preset condition includes at least one of the following two situations:

a situation where the quality of communication between the second wireless communication module 102 and the first wireless communication module 101 is lower than the quality of communication between the fourth wireless communication module 104 and the third wireless communication module 103; and

a situation where a distance between the at least one mobile monitoring device and the ward-level monitoring apparatus exceeds a first range.

It will be understood that in an embodiment of the disclosure, the processor 109 causes the wireless communication modulation module 201 to switch according to the preset condition, which can ensure that if the communication is performed in the first wireless transmission mode to cause poor quality of communication such that the transmitted patient recovery state parameter may be lost, the communication is switched to the second wireless transmission mode with better quality of communication to perform communication, so as to prevent the ward-level monitoring apparatus from being unable to monitor the target object, i.e., the patient, in real time due to the inability to receive the patient recovery state parameter.

It should be noted that in an embodiment of the disclosure, the processor 109 in the mobile monitoring device may control the wireless communication modulation module 201 to switch between the second wireless communication module 102 and the fourth wireless communication module 104, that is to say, different wireless transmission modes are implemented by means of controlling the wireless communication modulation module 201 to operate in different operating frequency bands. Of course, an independent second wireless communication module 102 and fourth wireless communication module 104 can also be configured so as to implement the first wireless transmission mode and the second wireless transmission method. It will be understood that the processor 109 may also directly control the switch between the second wireless communication module 102 and the fourth wireless communication module 104, without the need for the wireless communication modulation module 201.

As an example, in an embodiment of the disclosure, the first range is X, and the distance between the mobile monitoring device and the ward-level monitoring apparatus is Y. When Y is greater than X, the processor 109 in the mobile monitoring device sends a first instruction to the wireless communication modulation module 201, and the wireless communication modulation module 201 operates in the first operating frequency band according to the first instruction so as to form a second wireless communication module 102. Accordingly, when Y is less than or equal to X, the processor 109 in the mobile monitoring device sends a second instruction to the wireless communication modulation module 201, and the wireless communication modulation module operates in the second operating frequency band according to the second instruction so as to form a fourth wireless communication module 104.

In an embodiment of the disclosure, the second wireless communication module 102 operates in a medical professional frequency band not more than 1 GHz, and the fourth wireless communication module 104 is a WI-FI module.

The at least one mobile monitoring device transmits the patient recovery state parameter to the first wireless communication module 101 in the medical professional frequency band not more than 1 GHz by means of the second wireless communication module 102.

When a preset condition is satisfied, the at least one mobile monitoring device wirelessly transmits the patient recovery state parameter in a WI-FI mode by means of the fourth wireless communication module 104. The preset condition includes at least one of the following two situations:

a situation where the quality of communication between the second wireless communication module 102 and the first wireless communication module 101 is lower than the quality of communication between the fourth wireless communication module 104 and the third wireless communication module 103; and

a situation where a distance between the at least one mobile monitoring device and the ward-level monitoring apparatus exceeds a first range.

It will be understood that in an embodiment of the disclosure, the first wireless transmission mode may be specifically a WMTS transmission mode, and the second wireless transmission mode may be specifically a WI-FI transmission mode. That is to say, the at least one mobile monitoring device can transmit the patient recovery state parameter in the WMTS transmission mode and the WI-FI transmission mode. The specific selection of the mode for transmission may depend on the specific quality of communication in the two transmission modes, and may also depend on the specific distance between the at least one mobile monitoring device and the ward-level monitoring apparatus, so as to ensure the practical applications in different application scenarios, which is not limited in the embodiment of the disclosure.

It should be noted that in an embodiment of the disclosure, the WMTS transmission mode not only has low power, but also has better privacy. Therefore, the first wireless transmission mode may use a WMTS transmission mode to implement short-distance real-time transmission of the patient recovery state parameter. However, since the WMTS transmission mode has low power, the transmission distance is limited, only limited to the short-distance transmission. When long-distance transmission is required, the patient recovery state parameter can be transmitted in the second wireless transmission mode, i.e., the WI-FI transmission mode. That is to say, the first wireless transmission mode and the second wireless transmission mode can ensure the short-distance and long-distance transmission of the patient recovery state parameter.

In an embodiment of the disclosure, the patient recovery monitoring system further comprises: a hospital-level monitoring apparatus. The hospital-level monitoring apparatus is communicatively connected to the department-level monitoring apparatus, and the department-level monitoring apparatus transmits the patient recovery state parameter to the hospital-level monitoring apparatus.

Specifically, in an embodiment of the disclosure, as shown in FIG. 3, the apparatus at the previous level of the department-level monitoring apparatus is the hospital-level monitoring apparatus. After the department-level monitoring apparatus obtains the patient recovery state parameter, the patient recovery state parameter is transmitted to the hospital-level monitoring apparatus in a wired transmission mode, such that the monitoring apparatus of each level can obtain the patient recovery state parameter, which is applied to different actual scenarios.

In an embodiment of the disclosure, the patient recovery monitoring system further comprises: a hospital-level monitoring apparatus. The hospital-level monitoring apparatus is communicatively connected to the wireless transceiver, and the at least one mobile monitoring device accesses the wireless transceiver by means of the fourth wireless communication module 104, so as to transmit the patient recovery state parameter from the at least one mobile monitoring device to the hospital-level monitoring apparatus in the second wireless transmission mode by means of the wireless transceiver.

It should be noted that in an embodiment of the disclosure, as shown in FIG. 3, the hospital-level monitoring apparatus can not only obtain the patient recovery state parameter from the department-level monitoring apparatus as described above, but also directly obtain the patient recovery state parameter from the mobile monitoring device in the second wireless transmission mode. At this time, the hospital-level monitoring apparatus can be in wired connection with the wireless transceiver, and the mobile monitoring device 1 accesses the wireless transceiver, such that the patient recovery state parameter can be transmitted to the hospital-level monitoring apparatus in the B transmission mode by means of the wireless transceiver. The B transmission mode may be a WI-FI transmission mode, and may also be a WMTS transmission mode.

FIG. 11 is a structural schematic diagram V of a patient recovery monitoring system applied in a hospital according to an embodiment of the disclosure. As shown in FIG. 11, in an embodiment of the disclosure, at least one of the at least one mobile monitoring device further comprises: a mobile communication network module 202. The system further comprises: a hospital-level monitoring apparatus. The hospital-level monitoring apparatus is communicatively connected to the mobile communication network module 202 of the at least one mobile monitoring device by means of a mobile communication network, and the at least one mobile monitoring device obtains a patient recovery state parameter corresponding to the target object, and transmits the patient recovery state parameter to the hospital-level monitoring apparatus in a mobile communication network transmission mode by means of the mobile communication network module 202.

It should be noted that, in an embodiment of the disclosure, the mobile communication network module 202 may form a mobile communication network, such as a 2G/3G/4G/5G/NB-IoT/eMTC mobile network, that is, the hospital-level monitoring apparatus may also communicate with the at least one mobile monitoring device through the 2G/3G/4G/5G/NB-IoT/eMTC mobile network, so as to obtain the patient recovery state parameter.

Specifically, in an embodiment of the disclosure, when the patient is moving outside the hospital and needs to be monitored outside the hospital, the mobile monitoring device worn by the patient is configured with the mobile communication network module 202, and if the mobile monitoring device is in a coverage area of a 4G network base station, the mobile communication network module 202 automatically forms a 4G mobile network, and the patient recovery state parameter is transmitted to the hospital-level monitoring apparatus through the 4G mobile network. Of course, if the mobile monitoring device is in a coverage area of other network base station, the mobile communication network module 202 may also form the corresponding mobile network, to transmit the patient recovery state parameter. It will be understood that if the mobile monitoring device is in a coverage area of a plurality of network base stations, i.e., the mobile communication network module 202 may form a plurality of mobile networks, for example, when the mobile monitoring device is in a coverage area of a 3G network base station and a 4G network base station, a 3G mobile network may be formed and a 4G mobile network may also be formed, at this time, a mobile network with a higher signal intensity may be selected. It will be understood that, the mobile network may also be another mobile communication network, as long as it can perform wireless data transmission, which is not limited herein.

It should be noted that, in an embodiment of the disclosure, the same mobile communication network module 202 may also be configured in an ambulance or a bedside docking station, when the ambulance or the bedside docking station communicates with the mobile monitoring device and receives the patient recovery state parameters, the mobile communication network module 202 may further upload the received patient recovery state parameter to the hospital-level monitoring apparatus.

It should be noted that, in an embodiment of the disclosure, the mobile communication network module 202 may also be configured as an independent accessory, and is specifically integrated with a mobile monitoring device, which comprises the second wireless communication module 102 and the fourth wireless communication module 104, of the at least one mobile monitoring device. Of course, the mobile communication network module may also be integrated with any mobile monitoring device, so that when the at least one mobile monitoring device obtains the patient recovery state parameters, the mobile communication network module 202 forms the mobile network and uploads the parameter to the hospital-level monitoring apparatus.

It will be understood that, in an embodiment of the disclosure, the above three specific methods for the hospital-level monitoring apparatus to obtain the patient recovery state parameter are provided, specifically in practical applications, the corresponding communication mode may be selected according to the configuration of the hospital-level monitoring apparatus itself or the actual communication situation, which is not limited in the embodiment of the disclosure.

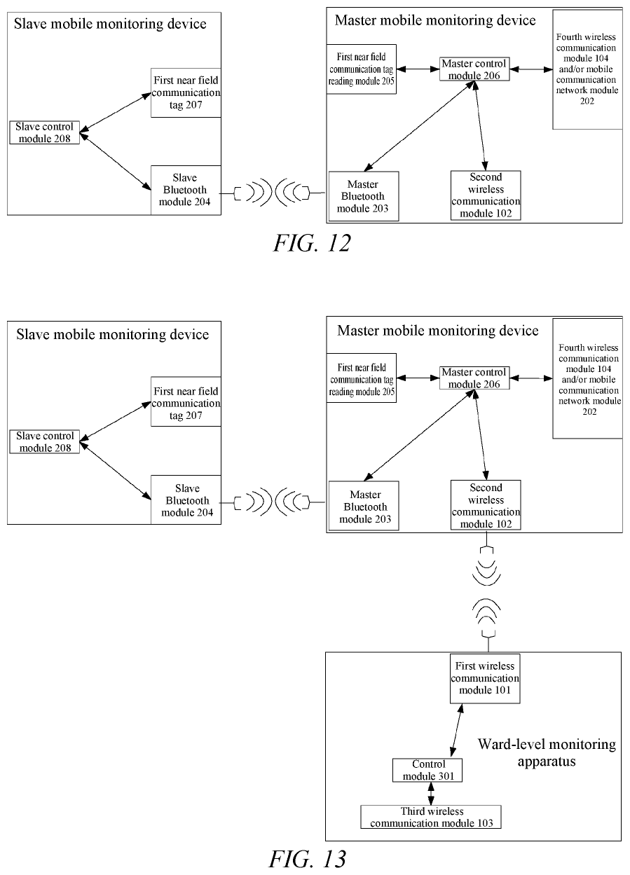

It should be noted that, in an embodiment of the disclosure, mobile monitoring devices included in the at least one mobile monitoring device may communicate with each other, and may be divided into a master mobile monitoring device and a slave mobile monitoring state. FIG. 12 is a communication schematic diagram of an exemplary master mobile monitoring device and slave mobile monitoring device according to an embodiment of the disclosure. The following is a detailed description based on FIG. 12.

In an embodiment of the disclosure, the at least one mobile monitoring device comprises at least one master mobile monitoring device, the master mobile monitoring device comprises the second wireless communication module 102, the master mobile monitoring device collects the patient recovery state parameter obtained from the at least one mobile monitoring device, and the second wireless communication module 102 transmits the patient recovery state parameter to the ward-level monitoring apparatus in the first wireless transmission mode.

As an example, in an embodiment of the disclosure, the master mobile monitoring device may be an ECG-PoD, which is used for collecting patient recovery state parameter, and the patient recovery state parameter is specifically obtained by the master mobile monitoring device and other mobile monitoring devices.

It should be noted that, in an embodiment of the disclosure, due to power consumption of the communication between the mobile monitoring device and the ward-level monitoring apparatus, if each mobile monitoring device worn by the patient communicates with the ward-level monitoring apparatus, the endurance time of each mobile monitoring device will be affected. In addition, if each mobile monitoring device worn by the patient communicates with the ward-level monitoring apparatus, a lot of spectrum resources will also be occupied. Therefore, in order to reduce power consumption and avoid occupying a lot of spectrum resources, the master mobile monitoring device is selected from the at least one mobile monitoring device, the master mobile monitoring device collects the patient recovery state parameter, and only the master mobile monitoring device communicates with the ward-level monitoring apparatus, i.e. transmitting the patient recovery state parameter. In actual use, since the ECG is usually an item that must be monitored, the ECG-Pod may be selected as the master mobile monitoring device.

In an embodiment of the disclosure, the at least one mobile monitoring device further comprises at least one slave mobile monitoring device, the master mobile monitoring device comprises a master Bluetooth module 203, and the slave mobile monitoring device comprises a slave Bluetooth module 204.

The patient recovery state parameter comprises a first patient recovery state parameter obtained by the master mobile monitoring device, and a second patient recovery state parameter obtained by the slave mobile monitoring device.

The slave mobile monitoring device transmits the second patient recovery state parameter to the master mobile monitoring device in a Bluetooth transmission mode by means of the slave Bluetooth module 204.

The master mobile monitoring device receives the second patient recovery state parameter by means of the master Bluetooth module 203.

It will be understood that, in an embodiment of the disclosure, the first patient recovery state parameter is obtained by the master mobile monitoring device, the second patient recovery state parameter is obtained by the slave mobile monitoring device, and the first patient recovery state parameter and the second patient recovery state parameter constitute the patient recovery state parameter.

As an example, in an embodiment of the disclosure, the master mobile monitoring device may be an ECG-PoD, and the slave mobile monitoring device may be a non-invasive blood pressure measurement device, i.e. an NIBP-PoD. The ECG-PoD may obtain the first patient recovery state parameter, i.e. ECG data, the NIBP-PoD may obtain the second patient recovery state parameter, i.e. blood pressure data, and the ECG-PoD may collect the blood pressure data and transmits same together with the ECG data to the ward-level monitoring apparatus. The slave mobile monitoring device NIBP-PoD uses an air pump to inflate the cuff to measure blood pressure, and compared with the traditional measurement method of an optical sensor, the accuracy is higher, and the requirement for blood pressure monitoring in the hospital may be met.

Specifically, in an embodiment of the disclosure, as shown in FIG. 12, the master Bluetooth module 203 of the master mobile monitoring device may perform Bluetooth transmission with the slave Bluetooth module 204 of the slave mobile monitoring device, so that the second patient recovery state parameter obtained by the slave mobile monitoring device is transmitted to the master mobile monitoring device, and the master mobile monitoring device collects the patient recovery state parameter.

It will be understood that, in an embodiment of the disclosure, before data transmission between the master mobile monitoring device and the slave mobile monitoring device is performed through Bluetooth, a Bluetooth connection between the two needs to be established.

In an embodiment of the disclosure, the master mobile monitoring device further comprises: a first near field communication tag reading module 205 and a master control module 206, and the slave mobile monitoring device further comprises: a first near field communication tag 207 and a slave control module 208.

The master mobile monitoring device reads the first near field communication tag 207 of the slave mobile monitoring device by means of the first near field communication tag reading module 205 to obtain a read signal.

The master mobile monitoring device further obtains Bluetooth connection information according to the read signal by means of the master control module 206, and controls the master Bluetooth module 203 to send a Bluetooth connection request to the slave Bluetooth module 204 according to the at least one Bluetooth connection information.

The slave mobile monitoring device controls the slave Bluetooth module 204 to establish a Bluetooth connection with the master Bluetooth module 203 by means of the slave control module 208.

It should be noted that, in an embodiment of the disclosure, when the master mobile monitoring device and the slave mobile monitoring device approach each other, the specific process of establishing the Bluetooth connection may be realized.

FIG. 13 is a communication schematic diagram of the master mobile monitoring device, the slave mobile monitoring device and the ward-level monitoring apparatus according to an embodiment of the disclosure. As shown in FIG. 13, with the Bluetooth transmission between the master mobile monitoring device and the slave mobile monitoring device, the master mobile monitoring device collects the patient recovery state parameter, and the patient recovery state parameter may be transmitted between the master mobile monitoring device and the ward-level monitoring apparatus in the first wireless transmission mode. The ward-level monitoring apparatus implements connection control and transmission control by means of a control module.

It will be understood that, in an embodiment of the disclosure, the master mobile monitoring device and the slave mobile monitoring device may of course also use other wireless communication modes to realize that the master mobile monitoring device collects the patient recovery state parameter, only the master mobile monitoring device and the slave mobile monitoring device are configured with corresponding functional modules. For example, the master mobile monitoring device and the slave mobile monitoring device may also communicate wirelessly through wireless transmission modes such as WI-FI and infrared.

It should be noted that, in an embodiment of the disclosure, as shown in FIGS. 12 and 13, the fourth wireless communication module 104 and/or the mobile communication network module 202 are/is arranged on the master mobile monitoring device.

It will be understood that, in an embodiment of the disclosure, the ward-level monitoring apparatus, the department-level monitoring apparatus and the hospital-level monitoring apparatus may be different types of apparatuses, and the specific ward-level monitoring apparatus, the specific department-level monitoring apparatus and the specific hospital-level monitoring apparatus are not limited in the embodiments of the disclosure.

It should be noted that, in an embodiment of the disclosure, the hospital-level monitoring apparatus may be a hospital-level data center/hospital-level emergency center, and the department-level monitoring apparatus may be a work station/central station. The specific hospital-level monitoring apparatus and the specific department-level monitoring apparatus are not limited in the embodiment of the disclosure.

In an embodiment of the disclosure, the ward-level monitoring apparatus comprises: any one of: a bedside monitor, a bedside docking station, a medical care bed on-board apparatus, a portable monitor, and a bedside routing apparatus.

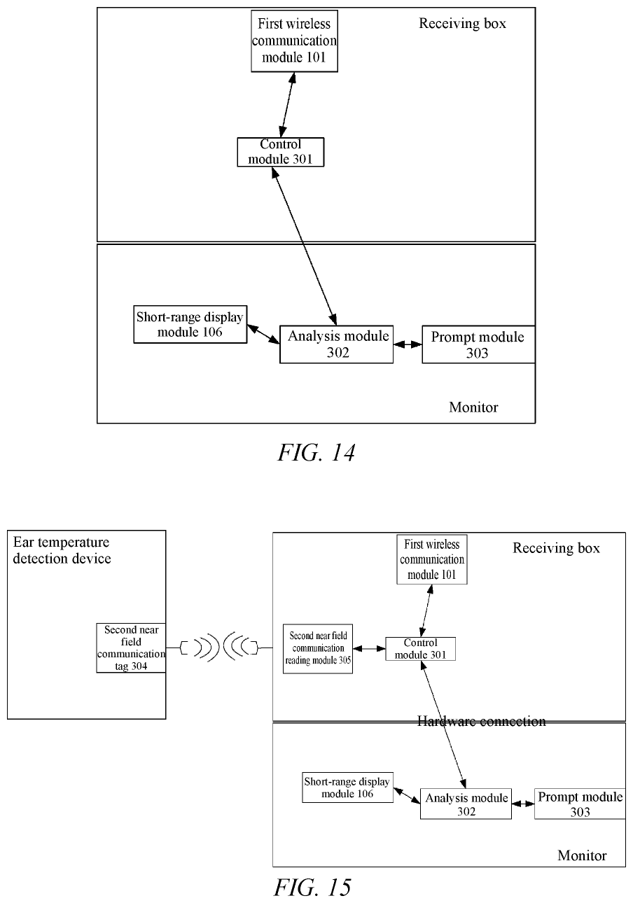

Specifically, in an embodiment of the disclosure, the ward-level monitoring apparatus is a bedside monitor, and FIG. 14 is a structural schematic diagram I of an exemplary bedside monitor according to an embodiment of the disclosure. As shown FIG. 14, the bedside monitor comprises a receiving box and a monitor, and the receiving box is detachably installed on the monitor; the first wireless communication module 101 is provided on the receiving box, and the monitor comprises a nearby display module 106.

The receiving box receives the patient recovery state parameter in the first wireless transmission mode by means of the first wireless communication module 102 to form a nearby display data and transmit same to the monitor by means of a hardware connected to the receiving box and the monitor.

The monitor displays the nearby display data by means of the nearby display module 106.

It should be noted that, in an embodiment of the disclosure, the monitor may further comprise an analysis module 302; and the monitor analyzes the patient recovery state parameter by means of the analysis module 302, and evaluates a recovery state of the target object.

It will be understood that, in an embodiment of the disclosure, the patient recovery state parameter may be used to analyze the target object, i.e. the recovery state of the patient, for example, the patient recovery state parameter that may be obtained from the monitor comprise heart rate, and if the heart rate always exceeds a preset heart rate threshold for a preset period of time, the recovery state of the target object is evaluated to be not good.

It should be noted that, in an embodiment of the disclosure, the monitor may further comprise a prompt module 303; and the monitor determines that there is an abnormality in the patient recovery state parameter, and prompts the abnormality according to a preset prompt mode by means of the prompt module 303.

It will be understood that, in an embodiment of the disclosure, the ward-level monitoring apparatus is a bedside monitor. Since the bedside monitor is fixed in the ward, with the structure shown in FIG. 14, the patient recovery state parameter may be received in the first wireless transmission mode and/or the second wireless transmission mode. In addition, the functions of data analysis, data display and alarm prompt may also be integrated to realize all-round monitoring of the patient.

Specifically, in an embodiment of the disclosure, the ward-level monitoring apparatus is a bedside docking station, and the bedside docking station comprises the first wireless communication module 101; and the bedside docking station receives the patient recovery state parameter in the first wireless transmission mode by means of the first wireless communication module 101.

It should be noted that, in an embodiment of the disclosure, the bedside docking station may be a plug-in box structure with a plurality of extended module slots beside the bed, which is usually hung beside the bed, and may be located on the bed or on the wall, or on a point-of-care concentrator station.

Specifically, in an embodiment of the disclosure, the ward-level monitoring apparatus is a bedside monitor, which comprises the first wireless communication module 101 and further comprises an extended function parameter module, and the extended function parameter module is detachably installed inside the bedside monitor.

The bedside monitor obtains an extended recovery state parameter by means of the extended function parameter module.

The bedside monitor obtains the patient recovery state parameter from the at least one mobile monitoring device with the first wireless communication module 301.

The bedside monitor combines the extended physiological parameter measurement data and the patient recovery state parameter to form a nearby display data, and outputs and displays the nearby display data on a nearby display module 106 of the bedside monitor, the nearby display data comprising a numerical value and/or a waveform.

Specifically, in an embodiment of the disclosure, the ward-level monitoring apparatus is a bedside monitor.

The bedside monitor obtains a ward round measurement data from a ward round monitor in a third wireless transmission mode.

The bedside monitor obtains the patient recovery state parameter from the at least one mobile monitoring device with the first wireless communication module 101.

The bedside monitor combines the ward round measurement data and the patient recovery state parameter to form a nearby display data, and outputs and displays the nearby display data on a nearby display module 106 of the bedside monitor, the nearby display data comprising a numerical value and/or a waveform.

It should be noted that, in an embodiment of the disclosure, the third wireless transmission mode may be a near field communication mode such as the Bluetooth transmission mode or a near field wireless communication mode, and the specific third wireless transmission mode is not limited in the embodiment of the disclosure.

Specifically, in an embodiment of the disclosure, the ward-level monitoring apparatus is a medical care bed on-board apparatus, which comprises the first wireless communication module 101 and a monitoring sensor.

The medical care bed on-board apparatus obtains a bed on-board physiological sign parameter by means of the monitoring sensor, and receives the patient recovery state parameter in the first wireless transmission mode by means of the first wireless communication module 101.

It should be noted that, in an embodiment of the disclosure, the monitoring sensor on the medical care bed on-board apparatus may be a non-contact sensor or a wired contact sensor, and the bed on-board physiological sign parameter obtained thereby may be a sleep detection parameter, etc. The medical care bed on-board apparatus may further comprise a concentrator station, and the concentrator station may provide a power or data receiving and processing terminal for the monitoring sensor.

It will be understood that, in an embodiment of the disclosure, the medical care bed on-board apparatus may specifically communicate wirelessly with the master mobile monitoring device in at least one mobile monitoring device, and receive the patient recovery state parameter in the first wireless transmission mode. Of course, the medical care bed on-board apparatus may also transmit the bed on-board physiological sign parameter to the master mobile monitoring device in the first wireless transmission mode, the master mobile monitoring device may be configured with a display module, and the display module may not only display the patient recovery state parameter, but also display the received bed on-board physiological sign parameter.

It should be noted that, in an embodiment of the disclosure, the medical care bed on-board apparatus further comprises: a nearby display module 106; and the medical care bed on-board apparatus forms a nearby display data according to the patient recovery state parameter and the bed on-board physiological sign parameter, and displays the nearby display data by means of the nearby display module 106.

As an example, in an embodiment of the disclosure, the monitoring sensor integrated in the hospital bed is specifically a fabric sensor, and can be used to detect the posture change of the patient on the hospital bed, so as to combine the patient recovery state parameter, such as an ECG signal, to jointly analyze sleep conditions.

It should be noted that, in an embodiment of the disclosure, the medical care bed on-board apparatus transmits the bed on-board physiological sign parameter to the second wireless communication module in the first wireless transmission mode by means of the first wireless communication module.

The at least one mobile monitoring device receives the bed on-board physiological sign parameter in the first wireless transmission mode by means of the second wireless communication module 102.

Specifically, in an embodiment of the disclosure, the ward-level monitoring apparatus is a portable monitor, which comprises the first wireless communication module 101 and the nearby display module 106.

The portable monitor receives the patient recovery state parameter in the first wireless transmission mode by means of the first wireless communication module 101 to form a nearby display data, and displays the nearby display data by means of the nearby display module 106.

Specifically, in an embodiment of the disclosure, the ward-level monitoring apparatus is a bedside routing apparatus, which comprises the first wireless communication module 101 and the third wireless communication module 103, and the system further comprises a department-level monitoring apparatus, which comprises the fourth wireless communication module 104.

The bedside routing apparatus receives the patient recovery state parameter in the first wireless transmission mode by means of the first wireless communication module 101, and transmits the patient recovery state parameter to the department-level monitoring apparatus in a second wireless transmission mode by means of the third wireless communication module 103.

The department-level monitoring apparatus receives the patient recovery state parameter in the second wireless transmission mode by means of the fourth wireless communication module 104.

It should be noted that, in an embodiment of the disclosure, the patient recovery monitoring system may further comprise at least one extended monitoring device comprising: a second near field communication tag 304. The ward-level monitoring apparatus comprises: a second near field communication tag reading module 305.

The ward-level monitoring apparatus reads the second near field communication tag 304 by means of the second near field communication tag reading module 305, to obtain an additional recovery state parameter obtained from the extended monitoring device.

FIG. 15 is a communication schematic diagram of an exemplary extended monitoring device according to an embodiment of the disclosure. As shown in FIG. 15, the extended monitoring device is specifically an ear temperature detection device, and the ward-level monitoring apparatus is a bedside monitor, which comprises: a receiving box and a monitor. The receiving box may further comprise a second near field communication tag reading module 305, and reads the second near field communication tag 304 of the ear temperature detection device by means of the second near field communication tag reading module 305, so as to directly obtain an ear temperature data obtained from the ear temperature detection device.

It should be noted that, in an embodiment of the disclosure, the first wireless transmission mode is a WMTS transmission mode. For the WMTS transmission mode, different frequencys also have the problem of mutual noise interference, i.e. adjacent channel interference, that is, at a certain frequency, the stronger the transmission power is, the noise generated at the nearby frequency is larger, thereby affecting the communication of the apparatus on the nearby frequency. As shown in FIG. 3, if the mobile monitoring device 1 and the mobile monitoring device 2 respectively transmit the patient recovery state parameter to the ward-level monitoring apparatus 11 in the WMTS mode, at the same time, the mobile monitoring device 3 and the ward-level monitoring apparatus 21 transmit the patient recovery state parameter in the WMTS mode, and when the transmission power of WMTS between the mobile monitoring device 1 and the ward-level monitoring apparatus 11 is too large, the transmission process of the mobile monitoring device 2 and even the mobile monitoring device 3 may be interfered. It will be understood that, in a practical application process, because the distance between most point-to-point communication apparatuses is relatively short, low power transmission may be used, and when the distance between the point-to-point communication apparatuses gradually increases or there is obstruction therebetween, in order to ensure the reliability of transmission, the transmission power between the mobile monitoring device and the ward-level monitoring apparatus may be appropriately increased within the allowable range. Therefore, in the monitoring system of the disclosure, dynamic power regulation for WMTS coverage can be performed. Specifically, the strategy of dynamic power regulation uses real-time monitoring of a signal-to-noise ratio, when the signal-to-noise ratio is lower than a first preset threshold, the mobile monitoring device and the ward-level monitoring apparatus increases transmission power, and when the signal-to-noise ratio is higher than a second preset threshold, in order to reduce interference to other apparatuses, the transmission power may be reduced accordingly, specifically the first preset threshold and the second preset threshold may be preset by the user, which is not specifically limited in the embodiment of the disclosure. Of course, in the WMTS transmission mode, when the distance between the ward-level monitoring apparatuses increases or there is obstruction therebetween, but it has not yet switched to the second wireless transmission mode, such as the WI-FI transmission mode, the mobile monitoring device may also, according to the increase of the distance, correspondingly increase the transmission power of the mobile monitoring device and the ward-level monitoring apparatus, to solve the problem of not being able to transmit the patient recovery state parameter.

Another embodiment of the disclosure provides another patient recovery monitoring system applied in a hospital, the system comprising:

a ward-level monitoring apparatus, a department-level monitoring apparatus and at least one mobile monitoring device, where

the at least one mobile monitoring device is capable of respectively transmitting a patient recovery state parameter with the ward-level monitoring apparatus and the department-level monitoring apparatus in a wireless transmission mode;

the at least one mobile monitoring device is wearable on a body of a target object, and is configured to obtain the patient recovery state parameter corresponding to the target object; and

the at least one mobile monitoring device transmits the patient recovery state parameter detected in real time to the ward-level monitoring apparatus in a first wireless transmission mode.

It should be noted that, in an embodiment of the disclosure, a transmission frequency band corresponding to the first wireless transmission mode is less than a transmission frequency band corresponding to the second wireless transmission mode.

It should be noted that, in an embodiment of the disclosure, the transmission frequency band corresponding to the first wireless transmission mode is not more than 1 GHz.

It should be noted that, in an embodiment of the disclosure, the first wireless transmission mode uses a medical professional frequency band, and the second wireless transmission mode uses a general wireless data transmission frequency band.

It should be noted that, in an embodiment of the disclosure, there is a data communication function between the ward-level monitoring apparatus and the department-level monitoring apparatus.

It should be noted that, in an embodiment of the disclosure, the at least one mobile monitoring device obtains a downlink data from the ward-level monitoring apparatus in the first wireless transmission mode, the downlink data including a control information, a handshake information, a measurement configuration, an interactive information, and a starting measurement item.

It will be understood that, in an embodiment of the disclosure, in the patient recovery monitoring system added in the department-level monitoring apparatus, the communication process between the at least one mobile monitoring device, the ward-level monitoring apparatus and the department-level monitoring apparatus, and communication modes of various combinations, have been detailed in the above content, which will not be repeated herein.

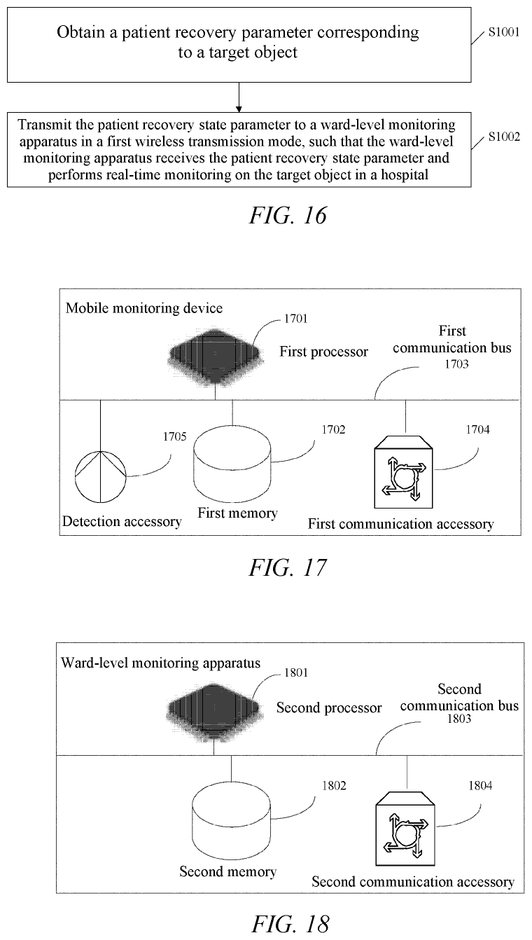

Still another embodiment of the disclosure provides a patient recovery monitoring method applied in a hospital, which is applied to at least one mobile monitoring device. FIG. 16 is a schematic flowchart of a patient recovery monitoring method according to an embodiment of the disclosure. As shown in FIG. 16, the method mainly comprises the following steps.

At S1601, a patient recovery parameter corresponding to a target object is obtained.

In an embodiment of the disclosure, at least one mobile monitoring device may obtain the patient recovery parameter corresponding to the target object, i.e. the patient.

It should be noted that, in an embodiment of the disclosure, the patient recovery parameter includes three types of parameters: parameters related to movement amount, such as movement steps, step frequency, movement distance, calories, etc.; a physiological parameter, such as blood oxygen, blood pressure, pulse rate, body temperature, ECG, respiration, and other parameters, as well as related statistics and a rate of change of these parameters; and time parameters of the human body state, for example, a movement-related or sleep-related time parameter that characterizes a human body state. The specific patient recovery state parameter is not limited in this embodiment of the disclosure.

As an example, in an embodiment of the disclosure, the at least one mobile monitoring device comprises: an ECG-PoD and an NIBP-PoD. The ECG-PoD may be used to obtain an ECG parameter, the NIBP-PoD may be used to obtain a blood pressure parameter, and the ECG parameter and the blood pressure parameter are both patient recovery state parameters.

It should be noted that, in an embodiment of the disclosure, the at least one mobile monitoring device comprises at least one master mobile monitoring device, and the at least one mobile monitoring device obtains the patient recovery state parameter corresponding to the target object, consisting in that the master mobile monitoring device collects the patient recovery state parameter obtained from the at least one mobile monitoring device.

Specifically, in an embodiment of the disclosure, the at least one mobile monitoring device further comprises at least one slave mobile monitoring device, the master mobile monitoring device comprises a master Bluetooth module 203, and the slave mobile monitoring device comprises a slave Bluetooth module 204; the patient recovery state parameter comprises a first patient recovery state parameter obtained by the master mobile monitoring device, and a second patient recovery state parameter obtained by the slave mobile monitoring device; the at least one mobile monitoring device uses the master mobile monitoring device to collect the patient recovery state parameter obtained from the at least one mobile monitoring device, consisting in that the slave Bluetooth module 204 of the slave mobile monitoring device transmits the second patient recovery state parameter to the master mobile monitoring device in the Bluetooth transmission mode; and the master Bluetooth module 203 of the master mobile monitoring device receives the second patient recovery state parameter. It will be understood that, the slave mobile monitoring device may also transmit the second patient recovery state parameter to the master mobile monitoring device in other transmission modes, and the other transmission modes include but are not limited to WI-FI transmission, data line transmission, etc.

It should be noted that, in an embodiment of the disclosure, the master mobile monitoring device further comprises: a first near field communication tag reading module 205 and a master control module 206, and the slave mobile monitoring device further comprises: a first near field communication tag 207 and a slave control module 208. Before the at least one mobile monitoring device transmits the second patient recovery state parameter to the master mobile monitoring device in the Bluetooth transmission mode by means of the slave Bluetooth module 204 of the slave mobile monitoring device, the method further comprises: reading the first near field communication tag 207 of the slave mobile monitoring device by the first near field communication tag reading module 205 of the master mobile monitoring device to obtain a read signal; obtaining a Bluetooth connection information according to the read signal by the master control module 206 of the master mobile monitoring device; controlling, by the master control module 206 of the master mobile monitoring device according to the at least one Bluetooth connection information, the master Bluetooth module 203 to send a Bluetooth connection request to the slave Bluetooth module 204; and controlling, by the slave control module 208 of the slave mobile monitoring device, the slave Bluetooth module 204 to establish a Bluetooth connection with the master Bluetooth module 203.

It should be noted that, in an embodiment of the disclosure, before the at least one mobile monitoring device transmits the patient recovery state parameter to the ward-level monitoring apparatus, the two need to be paired.

Specifically, in an embodiment of the disclosure, the ward-level monitoring apparatus comprises a first wireless communication module 101, and at least one of the at least one mobile monitoring device comprises a second wireless communication module 102 capable of being coupled to and communicating with the first wireless communication module 101. Before the at least one mobile monitoring device transmits the patient recovery state parameter to the ward-level monitoring apparatus in the first wireless transmission mode, the method further comprises: when the ward-level monitoring apparatus broadcasts a pairing code at a fixed frequency by means of the first wireless communication module 101, scanning the fixed frequency by means of the second wireless communication module 102 to receive the pairing code; and synchronously setting the fixed frequency according to the pairing code by means of the second wireless communication module 102, to realize the pairing with the ward-level monitoring apparatus.

Specifically, in an embodiment of the disclosure, the second wireless communication module comprises 102: a second radio-frequency module 1021 and a second single-chip microcomputer 1022. The at least one mobile monitoring device transmitting the patient recovery state parameter to the ward-level monitoring apparatus in the first wireless transmission mode comprises: transmitting the patient recovery state parameter to the second radio-frequency module 1021 by the second single-chip microcomputer 1022, and transmitting the patient recovery state parameter to the ward-level monitoring apparatus by the second radio-frequency module 1021.

At S1602, the patient recovery state parameter is transmitted to a ward-level monitoring apparatus in a first wireless transmission mode, such that the ward-level monitoring apparatus receives the patient recovery state parameter and performs real-time monitoring on the target object in the hospital.

In an embodiment of the disclosure, the at least one mobile monitoring device transmits the patient recovery state parameter to the ward-level monitoring apparatus, the medical staff may obtain the patient recovery state parameter from the ward-level monitoring apparatus in the process of moving the target object, i.e. the patient, so as to monitor the patient.

It should be noted that, in an embodiment of the disclosure, the at least one mobile monitoring device comprises at least one display module 105; and the at least one mobile monitoring device, after obtaining the patient recovery state parameter corresponding to the target object, may also form local display data according to the patient recovery state parameter, and outputs and displays the local display data by means of the display module 105.

It will be understood that, in an embodiment of the disclosure, the display module 105 outputs and displays the local display data, the medical staff may intuitively see that the local display data may be displayed in the form of numerical values or in the form of waveforms, specifically the local display data is not limited in the embodiment of the disclosure.

It will be understood that, in an embodiment of the disclosure, the ward-level monitoring apparatus, after obtaining the patient recovery parameters corresponding to the target object, may also transmit the patient recovery state parameter to the hospital-level monitoring apparatus in the second wireless transmission mode.

Yet still another embodiment of the disclosure provides a patient recovery monitoring method applied in a hospital, which is applied to a ward-level monitoring apparatus. The method mainly comprises the following step:

receiving a patient recovery state parameter corresponding to a target object from at least one mobile monitoring device in a first wireless transmission mode, so as to perform real-time monitoring on the target object in the hospital. The at least one mobile monitoring device is configured to obtain the patient recovery state parameter.

It should be noted that, in an embodiment of the disclosure, the ward-level monitoring apparatus comprises the first wireless communication module 101, and the at least one mobile monitoring device comprises the second wireless communication module 102 capable of being coupled to and communicating with the first wireless communication module 101. Before the ward-level monitoring apparatus receives the patient recovery state parameter corresponding to the target object from the at least one mobile monitoring device in the first wireless transmission mode, the method further comprises: broadcasting a pairing code at a fixed frequency by means of the first wireless communication module 101; and realizing the pairing with the at least one mobile monitoring device when the at least one mobile monitoring device scans the fixed frequency by means of the second wireless communication module 102 to receive the pairing code and synchronously sets the fixed frequency according to the pairing code.

It should be noted that, in an embodiment of the disclosure, the first wireless communication module 101 comprises: at least one first radio-frequency module 1011 and a first single-chip microcomputer 1012; The ward-level monitoring apparatus receiving the patient recovery state parameter corresponding to the target object from the at least one mobile monitoring device in the first wireless transmission mode comprises: receiving at least one patient recovery state parameter by the at least one first radio-frequency module 1011 to obtain at least one set of the patient recovery state parameters when the at least one mobile monitoring device transmits the patient recovery state parameter by means of the second wireless communication module 102; and selecting a set of data from the at least one set of the patient recovery state parameters by the first single-chip microcomputer 1012 so as to implement the real-time monitoring.

It should be noted that, in an embodiment of the disclosure, after the ward-level monitoring apparatus receives the patient recovery state parameter corresponding to the target object from the at least one mobile monitoring device in the first wireless transmission mode, the method further comprises: performing a communication connection with a hospital-level monitoring apparatus and/or a department-level monitoring apparatus, and transmitting the patient recovery state parameter to the hospital-level monitoring apparatus and/or the department-level monitoring apparatus.

It should be noted that, in an embodiment of the disclosure, the hospital-level monitoring apparatus may be a hospital-level data center/hospital-level emergency center, and the department-level monitoring apparatus may be a work station/central station. The specific hospital-level monitoring apparatus and the specific department-level monitoring apparatus are not limited in the embodiment of the disclosure.

It should be noted that, in an embodiment of the disclosure, the ward-level monitoring apparatus comprises: any one of: a bedside monitor, a bedside docking station, a medical care bed on-board apparatus, a portable monitor, and a bedside routing apparatus. The specific ward-level monitoring apparatus is not limited in the embodiment of the disclosure.

Yet another embodiment of the disclosure provides a mobile monitoring device. FIG. 17 is a schematic structure diagram of a mobile monitoring device according to an embodiment of the disclosure. As shown in FIG. 17, the mobile monitoring device comprises: a first processor 1701, a first memory 1702, a first communication bus 1703, a first communication accessory 1704 and a detection accessory 1705.

The first communication bus 1703 is configured to implement communication connections among the first processor 1701, the first memory 1702, the first communication accessory 1704 and the detection accessory 1705.

The first processor 1702, the first communication accessory 1704 and the detection accessory 1705 are configured to execute a first monitoring procedure stored in the first memory 1702, to implement the monitoring method applied to a mobile monitoring device.

Yet another embodiment of the disclosure provides a ward-level monitoring apparatus. FIG. 18 is a structural schematic diagram of a ward-level monitoring apparatus according to an embodiment of the disclosure. As shown in FIG. 18, the ward-level monitoring apparatus comprises: a second processor 1801, a second memory 1802, a second communication bus 1803 and a second communication accessory 1804.

The second communication bus 1803 is configured to implement communication connections among the second processor 1801, the second memory 1802 and the second communication accessory 1804.

The second processor 1801 and the second communication accessory 1804 are configured to execute a second monitoring procedure stored in the second memory 1802, to implement the monitoring method applied to a ward-level monitoring apparatus.

An embodiment of the disclosure provides a computer-readable storage medium storing a first monitoring procedure, which is executable by a first processor, a first communication accessory and a detection accessory to implement the monitoring method applied to a mobile monitoring device.

An embodiment of the disclosure provides a computer-readable storage medium storing a second monitoring procedure, which is executable by a second processor and a second communication accessory to implement the patient recovery monitoring method applied to a ward-level monitoring apparatus.

It should be noted that the computer-readable storage medium may be a volatile memory, such as a Random-Access Memory (RAM), or a non-volatile memory, such as a Read-Only Memory (ROM), a flash memory, a Hard Disk Drive (HDD) or a Solid-State Drive (SSD); or a respective device comprising one or any combination of the above memories, such as a mobile phone, a computer, a tablet, and a personal digital assistant.

Those skilled in the art should understand that the embodiments of the disclosure may be provided as a method, a system, or a computer program product. Therefore, the disclosure may take the form of hardware embodiments, software embodiments, or embodiments with a combination of software and hardware. Moreover, the disclosure may take the form of a computer program product that is implemented on one or more computer-usable storage media (including but not limited to a disk memory and an optical memory, etc.) that comprise computer-usable program codes.

The disclosure is described with reference to flow charts and/or block diagrams of the methods, apparatuses (systems), and computer program products according to the embodiments of the disclosure. It should be understood that each procedure and/or block in the flow charts and/or block diagrams, and combinations of the procedures and/or blocks in the flow charts and/or block diagrams may be implemented by computer program instructions. These computer program instructions may be provided to a processor of a general-purpose computer, a special-purpose computer, an embedded processor or other programmable signal processing apparatuses to create a machine, such that the instructions executed by the processor of the computer or other programmable signal processing apparatuses create a device for implementing functions specified in one or more procedures in the flow charts and/or one or more blocks in the block diagrams.

These computer program instructions may also be stored in a computer-readable memory that may direct the computer or other programmable signal processing apparatus to operate in a specific manner, such that the instructions stored in the computer-readable memory create an article of manufacture including an instruction device, and the instruction device implements the functions specified in one or more procedures of the flow charts and/or one or more blocks of the block diagrams.

These computer program instructions may also be loaded onto the computer or other programmable signal processing apparatuses, such that a series of operation steps are executed on the computer or other programmable apparatuses to perform computer-implemented processing, and thus the instructions executed on the computer or other programmable apparatuses provide steps for implementing the functions specified in one or more procedures of the flow charts and/or one or more blocks of the block diagrams.

The above description is only certain embodiments of the disclosure, and is not intended to limit the scope of protection of the disclosure.

### INDUSTRIAL APPLICABILITY

In a technical solution of an embodiment of the disclosure, a patient recovery monitoring system applied in a hospital comprises: a ward-level monitoring apparatus comprising at least a first wireless communication module; and at least one mobile monitoring device wearable on a body of a target object, where at least one of the at least one mobile monitoring device comprises a second wireless communication module capable of being coupled to and communicating with the first wireless communication module, and the at least one mobile monitoring device obtains a patient recovery state parameter corresponding to the target object, and transmits the patient recovery state parameter to the first wireless communication module in a first wireless transmission mode by means of the second wireless communication module; and the ward-level monitoring apparatus receives the patient recovery state parameter from the at least one mobile monitoring device in the first wireless transmission mode by means of the first wireless communication module, so as to perform real-time monitoring on the target object in the hospital. That is to say, when a patient is moving outside the ward, a patient recovery state parameter may be obtained by a mobile monitoring device, and the patient recovery state parameter is transmitted to a ward-level monitoring apparatus, such that medical care personnel can check and learn the relevant condition of the patient at any time to realize the monitoring of the patient.

1. A patient recovery monitoring system applied to a hospital, the system comprising:
a ward-level monitoring apparatus comprising at least a first wireless communication module; and
at least one mobile monitoring device wearable on a body of a target object, wherein the at least one mobile monitoring device comprises a second wireless communication module capable of being coupled to and communicating with the first wireless communication module, and the at least one mobile monitoring device is configured to obtain a patient recovery state parameter of the target object, and to transmit the patient recovery state parameter to the first wireless communication module in a first wireless transmission mode by means of the second wireless communication module; and
the ward-level monitoring apparatus is configured to receive the patient recovery state parameter from the at least one mobile monitoring device in the first wireless transmission mode by means of the first wireless communication module, so as to perform real-time monitoring on the target object in the hospital.

2. The system of claim 1, wherein the at least one mobile monitoring device is wearable on one or more of a wrist, a leg, an arm, a chest, a finger, and a waist of the target object.

3. The system of claim 1, further comprising: a wireless transceiver, wherein
the ward-level monitoring apparatus further comprises a third wireless communication module capable of accessing the wireless transceiver;
the at least one mobile monitoring device further comprises a fourth wireless communication module capable of accessing the wireless transceiver; and
the ward-level monitoring apparatus is further configured to access the wireless transceiver by means of the third wireless communication module, and the at least one mobile monitoring device is further configured to access the wireless transceiver by means of the fourth wireless communication module, so as to transmit the patient recovery state parameter from the at least one mobile monitoring device to the ward-level monitoring apparatus in a second wireless transmission mode by means of the wireless transceiver, wherein a transmission frequency band corresponding to the first wireless transmission mode is smaller than a transmission frequency band corresponding to the second wireless transmission mode.

4. The system of claim 3, further comprising: at least one of a department-level monitoring apparatus and a hospital-level monitoring apparatus, wherein
the ward-level monitoring apparatus is capable of transmitting the patient recovery state parameter to the at least one of the department-level monitoring apparatus and the hospital-level monitoring apparatus by means of a built-in wired communication module; and/or
the at least one of the department-level monitoring apparatus and the hospital-level monitoring apparatus is communicatively connected to the wireless transceiver, and the at least one mobile monitoring device is further configured to access the wireless transceiver by means of the fourth wireless communication module, so as to transmit the patient recovery state parameter from the at least one mobile monitoring device to the at least one of the department-level monitoring apparatus and the hospital-level monitoring apparatus in the second wireless transmission mode by means of the wireless transceiver.

5. The system of claim 1, wherein the at least one mobile monitoring device is connected to an external parameter measurement accessory in a wired or wireless mode, and the external parameter measurement accessory is attached to a part of the body of the target object that corresponds to a parameter to be measured.

6. The system of claim 1, wherein the at least one mobile monitoring device further comprises at least one display module, and the ward-level monitoring apparatus further comprises at least one nearby display module;
the at least one mobile monitoring device is further configured to obtain the patient recovery state parameter of the target object to form local display data, and to output and display the local display data on the display module of the at least one mobile monitoring device, the local display data comprising a first numerical value and/or a first waveform; and
the ward-level monitoring apparatus is further configured to obtain the patient recovery state parameter of the target object by means of the second wireless communication module to form nearby display data, and to output and display the nearby display data on the nearby display module of the ward-level monitoring apparatus, the nearby display data comprising a second numerical value and/or a second waveform.

7. The system of claim 1, wherein the at least one mobile monitoring device further comprises at least one display module, and the system further comprises a department-level monitoring apparatus comprising at least one remote display module;
the at least one mobile monitoring device is further configured to obtain the patient recovery state parameter of the target object to form local display data, and to output and display the local display data on the display module of the at least one mobile monitoring device, the local display data comprising a first numerical value and/or a first waveform; and
the department-level monitoring apparatus is configured to obtain the patient recovery state parameter of the target object in a second wireless transmission mode to form remote display data, and to output and display the remote display data on the remote display module of department-level monitoring apparatus, the remote display data comprising a second numerical value and/or a second waveform.

8. The system of claim 1, wherein the first wireless transmission mode is configured to adopt a medical professional frequency band.

9. The system of claim 3, wherein the at least one mobile monitoring device further comprises a processor and a wireless communication modulation module,
the processor is configured to cause the wireless communication modulation module to operate in a first operating frequency band to form the second wireless communication module, such that the at least one mobile monitoring device transmits the patient recovery state parameter to the first wireless communication module in the first operating frequency band by means of the second wireless communication module; and wherein,
when a preset condition is satisfied, the processor is further configured to cause the wireless communication modulation module to switch to operate in a second operating frequency band to form the fourth wireless communication module, such that the at least one mobile monitoring device transmits the patient recovery state parameter in the second operating frequency band by means of the fourth wireless communication module, wherein the second operating frequency band is greater than the first operating frequency band, and wherein the preset condition is at least one of the following situations:
a quality of communication between the second wireless communication module and the first wireless communication module is lower than a quality of communication between the fourth wireless communication module and the third wireless communication module; and
a distance between the at least one mobile monitoring device and the ward-level monitoring apparatus exceeds a first range.

10. The system of claim 3, wherein the second wireless communication module is configured to operate in a medical professional frequency band and the fourth wireless communication module is a WI-FI module;
the at least one mobile monitoring device is further configured to transmit the patient recovery state parameter to the first wireless communication module in the medical professional frequency band by means of the second wireless communication module; and
when a preset condition is satisfied, the at least one mobile monitoring device is further configured to transmit the patient recovery state parameter in a WI-FI mode by means of the fourth wireless communication module, wherein the preset condition is at least one of the following situations:
a quality of communication between the second wireless communication module and the first wireless communication module is lower than a quality of communication between the fourth wireless communication module and the third wireless communication module; and
a distance between the at least one mobile monitoring device and the ward-level monitoring apparatus exceeds a first range.

11-12. (canceled)

13. The system of claim 1, wherein the at least one mobile monitoring device further comprises a mobile communication network module, and the system further comprises a hospital-level monitoring apparatus communicatively connected to the mobile communication network module of the at least one mobile monitoring device by means of a mobile communication network, and the at least one mobile monitoring device is further configured to obtain the patient recovery state parameter of the target object, and to transmit the patient recovery state parameter to the hospital-level monitoring apparatus in a mobile communication network transmission mode by means of the mobile communication network module.

14. The system of claim 3, wherein the wireless transceiver includes one of the following:
a WI-FI wireless transceiver; and
a wireless transceiver operating in a medical professional frequency band greater than the transmission frequency band corresponding to the first wireless transmission mode.

15. The system of claim 1, wherein the at least one mobile monitoring device further comprises a master mobile monitoring device, wherein the second wireless communication module is provided on the master mobile monitoring device, and
the master mobile monitoring device is configured to transmit the patient recovery state parameter to the ward-level monitoring apparatus in the first wireless transmission mode by means of the second wireless communication module.

16. (canceled)

17. The system of claim 16, wherein the master mobile monitoring device further comprises a near field communication tag reading module and a first control module, and the slave mobile monitoring device further comprises a near field communication tag and a second control module;
the master mobile monitoring device is further configured to read the near field communication tag of the slave mobile monitoring device by means of the near field communication tag reading module to obtain a read signal;
the master mobile monitoring device is further configured to obtain a Bluetooth connection information according to the read signal by means of the first control module, and to control the first Bluetooth module to send a Bluetooth connection request to the second Bluetooth module according to at least part of the Bluetooth connection information; and
the slave mobile monitoring device is further configured to control the second Bluetooth module to establish a Bluetooth connection with the first Bluetooth module by means of the second control module.

18. The system of claim 16, wherein the ward-level monitoring apparatus further comprises a third Bluetooth module; wherein
when a distance between the master mobile monitoring device and the ward-level monitoring apparatus is within a second range, the master mobile monitoring device is further configured to transmit the patient recovery state parameter to the ward-level monitoring apparatus in the Bluetooth transmission mode by means of the first Bluetooth module; and
the ward-level monitoring apparatus is further configured to receive the patient recovery state parameter by means of the third Bluetooth module.

19. (canceled)

20. The system of claim 1, wherein the ward-level monitoring apparatus is further configured to broadcast identification information of the ward-level monitoring apparatus at a fixed frequency by means of the first wireless communication module; and
the at least one mobile monitoring device is further configured to scan the fixed frequency by means of the second wireless communication module to receive the identification information, and to synchronously set the fixed frequency according to the identification information, so as to realize pairing with the ward-level monitoring apparatus.

21-22. (canceled)

23. The system of claim 1, wherein the ward-level monitoring apparatus further includes any one of a bedside monitor, a bedside docking station, a medical care bed on-board apparatus, a portable monitor, and a bedside routing apparatus.

24. The system of claim 1, wherein the ward-level monitoring apparatus is a bedside monitor which comprises a receiving box and a monitor, wherein the receiving box is detachably installed on the monitor, the first wireless communication module is provided on the receiving box, and the monitor comprises a nearby display module;
the receiving box is configured to receive the patient recovery state parameter in the first wireless transmission mode by means of the first wireless communication module to form nearby display data, and to transmit the nearby display data to the monitor by means of a hardware which connects the receiving box with the monitor; and
the monitor is configured to display the nearby display data by means of the nearby display module.

25-57. (canceled)

58. The system of claim 15, wherein the at least one mobile monitoring device further comprises a slave mobile monitoring device;
the patient recovery state parameter comprises a first patient recovery state parameter obtained by the master mobile monitoring device and a second patient recovery state parameter obtained by the slave mobile monitoring device;
the slave mobile monitoring device is configured to transmit the second patient recovery state parameter to the master mobile monitoring device; and
the master mobile monitoring device is further configured to receive the second patient recovery state parameter from the slave mobile monitoring device.

59. The system of claim 58, wherein the master mobile monitoring device comprises a first Bluetooth module, and the slave mobile monitoring device comprises a second Bluetooth module;
the slave mobile monitoring device is further configured to transmit the second patient recovery state parameter to the master mobile monitoring device in a Bluetooth transmission mode by means of the second Bluetooth module; and
the master mobile monitoring device is further configured to receive the second patient recovery state parameter from the slave mobile monitoring device by means of the first Bluetooth module.
